(19) United States
(12) Patent Application Publication
(10) Pub. No.: US 2003/0014004 A1
(43) Pub. Date: Jan. 16, 2003

(54) Insufflation gas conditioning apparatus and conditioning arrangement

(76) Inventor: Uwe Dey, Falkensee (DE)

Correspondence Address: VENABLE, BAETJER, HOWARD AND CIVILETTI, LLP, P.O. BOX 34385, WASHINGTON, DC, 20043-9998, US

(21) Appl. No.: 10/191,503

(22) Filed: Jul. 10, 2002

Related U.S. Application Data
(60) Provisional application No. 60/303,773, filed on Jul. 10, 2001.

(51) Int. Cl. A61M 37/00

(52) U.S. Cl. 604/26

(57) ABSTRACT

An insufflation gas conditioning apparatus is provided for use with an insufflation device. The insufflation apparatus comprises a humidifying and filtering component realized as a separate component from the insufflation device and having an output side connection for an insufflation hose. The component is connectable on or close to an insufflation gas output of the insufflation device.

### DESCRIPTION

The invention relates to an insufflation gas conditioning apparatus according to the preamble of claim 1, and to an insufflation arrangement according to the preamble of claim 3 comprising an insufflation gas conditioning apparatus of this type.

For minimal-invasive surgical operations with insufflation of the respective body cavity or the respective organ, in which higher insufflation gas flow rates are required, heating up the insufflation gas has been accepted to a large extent during the past years. Insufflation devices including heater means are, for example, known from U.S. Pat. No. 5,006,109.

From EP 0 564 953 A1, a heatable insufflation hose is also known, which is intended to be connected to a conventional insufflation apparatus not equipped with a heater means. Moreover, an arrangement for heating insufflation gas is known from WO 94/28952, which arrangement includes a heater at least in part incorporated within the insufflation instrument.

Apart from heating up the insufflation gas, a further conditioning, in particular the humidification of same, has been focussed on by the medical profession as a precautionary measure against an excessive tissue exsiccation during operations of a longer duration, and has become the object of technical development. Solutions of this kind are known from the Applicant's document DE 195 10 710 A1, as well as from U.S. Pat. No. 5,411,474 and U.S. Pat. No. 6,068,609.

These known device solutions for the insufflation gas conditioning, however, are still in need of improvement for certain fields of application with respect to the constructional laboriousness and implementation properties.

The invention therefore is based on the object of providing an improved insufflation gas conditioning apparatus, as well as an overall arrangement built-up on same permitting, in a flexible manner according to the users' premises, a simple and cost-efficient upgrading of existing insufflation devices, and which is of a simple structure and easy manipulation.

This task is solved by means of an insufflation gas conditioning apparatus having the features of claim 1, and an insufflation gas conditioning arrangement having the features of claim 3. Purposeful configurations of the inventive idea result from the dependent claims.

The invention embraces the essential idea of providing, turning away from the hitherto realized arrangement of a humidification and filter means for the insufflation gas at the distal end of the insufflation hose or distributed at various spots thereof, a joint arrangement of these means close to the output of the insufflation apparatus, hence in the proximal zone of the insufflation hose. This facilitates the manipulation of the arrangement and results in advantages with respect to the medical sterilization and, if the case may be, the possible re-use of the conditioning substances.

In one preferred embodiment, the conditioning apparatus may be directly connected to the insufflation apparatus via a standard connection means or an adapter, whereas its output comprises a hose connection piece for connecting the insufflation hose. A realization, however, is also possible, in which the conditioning apparatus is connected with the output of the insufflation apparatus via a first hose piece of a shorter length.

With respect to the specific configuration of the humidification and filtering means, reference is in particular made to DE 195 107 10 A1, the disclosure of which herewith will be included into that of the present application.

The cold due to evaporation arising during the humidification of the insufflation gas, preferably is compensated by a pre-heating of the gas performed either in the insufflation apparatus itself or at least by first heater means arranged on the input side of the insufflation gas conditioning apparatus. Hereby, in a first alternative, an (in particular) electric heater installed specifically for pre-heating purposes is concerned, in a second alternative, however, the waste heat of the apparatus itself—in particular of the electric and/or electronic components or of a solenoid valve or such like of same—is exploited.

The final adjustment of the insufflation gas temperature preferably ensues by means of second heater means arranged within the conditioning apparatus or on the output side thereof, and which comprise in particular an electric heater having a temperature control. As second heater means, the solutions known per se of the heatable insufflation hose or of the heated insufflation instrument may be used, a separate heater housing, however, may be provided on the output side of the heater housing receiving the actual insufflation gas conditioning apparatus (humidification and filter means).

Subsequently, the invention will be described in more detail by means of two preferred embodiments with reference to the drawings. These show:

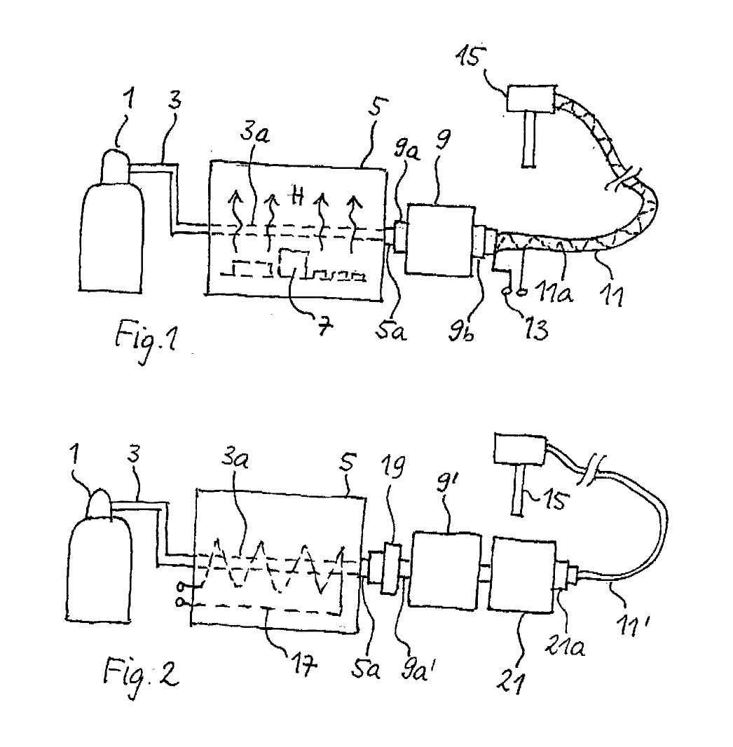

FIG. 1 a basic diagram of an insufflation arrangement including an insufflation gas conditioning apparatus and a heatable insufflation hose, and

FIG. 2 a basic diagram of an insufflation arrangement comprising an insufflation apparatus including a heater means, an insufflation gas conditioning apparatus, and a further separate heater means.

The insufflation arrangement as per FIG. 1 comprises an insufflation gas reservoir 1, an insufflation line 3 connected to same and leading to an insufflation device 5 of a constructional type known per se, an insufflation gas conditioning apparatus 9 connected to an insufflation gas output 5a of the insufflation device 5, a heatable insufflation hose 11 connected with the output of the insufflation gas conditioning apparatus 9, and an insufflation instrument 15 connected with the insufflation hose 11. The insufflation line 3 has an inner line portion 3a running within the device interior of the insufflation device 5, which line portion 3a is heated up by heat H ascending from electric and electronic components 7 of the insufflation device 5. Hereby, as well, a heating of the insufflation gas flowing through this line portion takes place.

The insufflation gas conditioning apparatus 9 has an input-side standard connection means 9a, the configuration of which being adapted to the insufflation gas output 5a of the insufflation device. The insufflation gas conditioning apparatus comprises, in a manner known per se from DE 195 10 710 C2, humidification means for adapting the humidity of the insufflation gas and/or the filtering means for reducing the impurity content of the gas. Said insufflation gas conditioning apparatus is connected with the insufflation hose again via a standardized output connection means 9b. Said insufflation hose 11 comprises an integrated heating coil 11a that is driven by a heating current source 13 and causes a further heating of the insufflation gas up to a value suited for being introduced into a body cavity of a patient.

The insufflation arrangement as per FIG. 2 comprises parts to some extent corresponding to the arrangement as per FIG. 1, which parts are designated with the same reference numerals and here are not discussed again. Said insufflation arrangement differs from that as per FIG. 1, for one, by a deviating realization of the insufflation device 5. Same namely comprises here an internal electric heating 17 for the controlled heating of the insufflation gas and which is illustrated in the Figure only schematically. Moreover, an adapter 19 is provided in this embodiment between an output connection means 5a of the insufflation device 5, which output connection means is realized according to a determined standard, and the input connection means 9′ not corresponding to said standard.

Finally, a separate heating module 21 is connected on the output side of the insufflation gas conditioning apparatus, the output 21a of which heating module 21 in the end being connected to a conventional insufflation hose 11′. The latter, in contrast to the realization as per FIG. 1, does not feature an integrated heater means and therefore is considerably cheaper and easier to manipulate due to its flexibility. Said heating module 21 preferably comprises a heating device configured as electric heating coil and associated to a line portion conducting the insufflation gas, a temperature sensor associated to said line portion, and an electronic control unit for driving the heating device in response to a temperature sensor signal, by means of which electronic control unit a pre-selected insufflation gas temperature is maintained constant. Controlled heating devices of this kind are known as such to the skilled person, so that a more detailed description can be omitted here.

The realization of the invention is not restricted to the mentioned examples but rather is likewise possible in a plurality of modifications that reside within the scope of the skilled person's activity.

1. An insufflation gas conditioning apparatus including humidification and filtering means, intended for being used with an insufflation device, which in particular comprises a pressure gas reservoir,
characterized by
the realization as a separate component having an output-side connection means for an insufflation hose, which component is connectable on or close to an insufflation gas output of the insufflation device.

2. The insufflation gas conditioning apparatus of claim 1,
characterized by
an input-side standard connection means or adapter for the direct connection with the insufflation gas output of the insufflation device.

3. An insufflation arrangement including an insufflation device, an insufflation gas conditioning apparatus according to claim 1, an insufflation hose connected to the output of the insufflation gas conditioning apparatus, and an insufflation instrument connected to the distal end of the insufflation hose,
characterized in that
on the input side of the insufflation gas conditioning apparatus, first heater means are provided for pre-heating the insufflation gas, and on the input or output side of the insufflation gas conditioning apparatus, second heater means are provided for the final temperature adjustment of the insufflation gas.

4. The insufflation arrangement of claim 3,
characterized in that
said second heater means are arranged in a separate housing, which is connected on the output side of the insufflation gas conditioning apparatus.

5. The insufflation arrangement of claim 3,
characterized in that
said second heater means are arranged in the insufflation hose or the insufflation instrument.

6. The insufflation arrangement of claim 3,
characterized in that
as the first heater means, electric and/or electronic components and/or a solenoid valve of the insufflation device are exploited.

7. The insufflation arrangement of claim 3,
characterized in that
said first heater means are formed by an in particular electric heating apparatus in the insufflation device.
